(19) United States
(12) Patent Application Publication
(10) Pub. No.: US 2023/0150932 A1
(43) Pub. Date: May 18, 2023

(54) COMPOSITIONS FOR STABILIZING AN ISOTHIOCYANATE

(71) Applicant: VIO CHEMICALS AG, Zurich (CH)

(72) Inventors: V. Theocharis KOFTIS, Thermi (GR); Efstratios NEOKOSMIDIS, Thermi (GR); L. Petros GZIKIS, Thermi (GR); Sakellarios TRAKOSSAS, Thermi (GR); Savvas THYSIADIS, Thermi (GR); Theodoros PANAGIOTIDIS, Thermi (GR); Thanos AANDREOU, Thermi (GR); Elli VASTARDI, Thermi (GR); Evanthia TOLIKA, Thermi (GR); Foteini ATHANASIADOU, Thermi (GR)

(21) Appl. No.: 17/918,821

(22) PCT Filed: Apr. 21, 2021
(86) PCT No.: PCT/EP2021/060461

§ 371 Date: Oct. 13, 2022

(30) Foreign Application Priority Data
Apr. 23, 2020 (WO) PCT/EP2020/025185
Jun. 30, 2020 (WO) PCT/EP2020/025306

(51) Int. Cl. C07C 331/20; A61K 8/46; A61K 8/36; A61K 31/26; A61K 47/12; A23L 33/105; A23L 33/115

(52) U.S. Cl. CPC C07C 331/20; A61K 8/46; A61K 8/361; A61K 31/26; A61K 47/12; A23L 33/105; A23L 33/115

(57) ABSTRACT

The present invention relates to stable compositions comprising an isothiocyanate. It also relates to methods for stabilizing an isothiocyanate. According to the invention, sulforaphane is stabilized in the presence of fatty acids. Compositions prepared by mixing sulforaphane with certain amounts of a fatty acid or a mixture of fatty acids exhibit a stabilizing effect with respect to residual sulforaphane. It was found that such compositions can maintain sulforaphane, as expressed by % w/w, more effectively, i.e. stabilize it, compared to neat sulforaphane, under certain storage conditions. This facilitates the distribution of sulforaphane as a component to be used for industrial purposes, for example in the preparation of various products, dietary supplements, food additives, pharmaceuticals, nutraceuticals, cosmetics etc, because it makes long-term storage easier and efficient. No complex techniques are required for the stabilization, such as encapsulation or complexation, neither extremely low temperatures.

### TECHNICAL FIELD OF THE INVENTION

The present invention relates to stable compositions comprising an isothiocyanate. It also relates to methods for stabilizing an isothiocyanate.

### BACKGROUND OF THE INVENTION

Sulforaphane [4-(methylsulfinyl)butylisothiocyanate] was isolated in 1992 by American researchers from the University of Johns Hopkins (Zhang et al., 1992). Sulforaphane is the product of the enzymatic hydrolysis of glucoraphanin [4-(methylsulfinyl)butyl glucosinolate] which is the most represented glucosinolate in broccoli. Several studies have shown that sulforaphane is sensitive to light, oxygen and temperature (Chiang et al., 1998; Yi Jin et al., 1999). This hampers the manufacturing and distribution of products and compositions comprising sulforaphane. Pure sulforaphane obtained as a normal chemical product (Cas No 142825-10-3) is to be kept at −20° C.

The prior art suggests various methods for the stabilization of sulforaphane.

In FR2888235 sulforaphane is stabilized by means of encapsulating into acacia gum. The proposed method is allegedly advantageous, because it allows the encapsulated composition to be converted into dry powder, which is disclosed as being more stable (p. 7, In. 13-15). The sulforaphane encapsulated in FR2888235 originates from extraction of broccoli seeds. The compositions prepared in FR2888235 comprise sulforaphane in concentrations of 36.05 and 22.4% w/w (tables 3-5). The compositions comprising the higher sulforaphane concentration exhibit less stability.

US2014170218A discloses an extraction method that provides a non-aqueous extract from Brassica Oleracea Italica, with low content of erucic acid and lipids, containing at least 30% by weight of Sulforaphane. This non-aqueous extract is stabilized by means of encapsulation into a polysaccharide matrix selected from acacia gum, maltodextrin and mixture thereof, leading to a powder containing at least 10% by weight of sulforaphane ([0036]). No further data are provided with respect to the stabilization effect. The suggested solution for stabilizing sulforaphane is limiting, since the produced encapsulated composition is only used as such in preparing further nutraceutical compositions. Furthermore, it is not apparent if and how higher concentrations of sulforaphane can be reached by this method or if it is applicable in sulforaphane of synthetic origin.

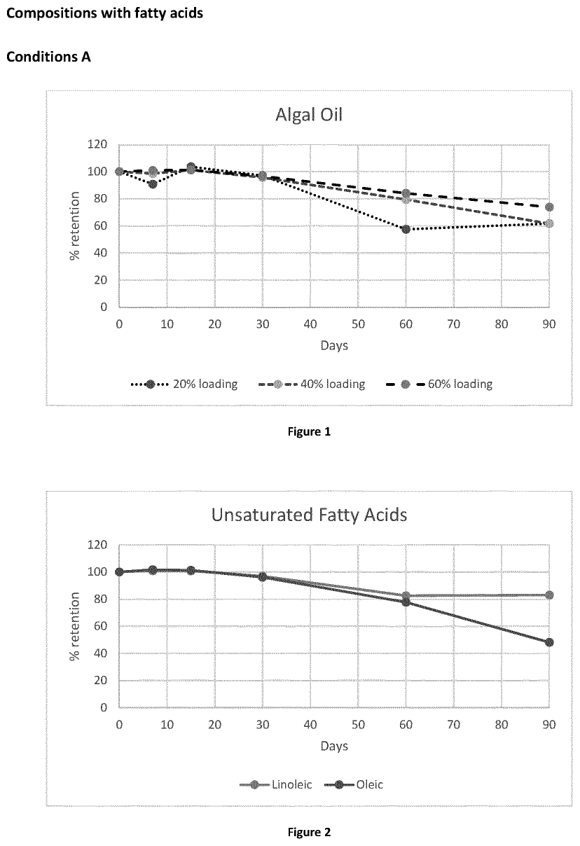

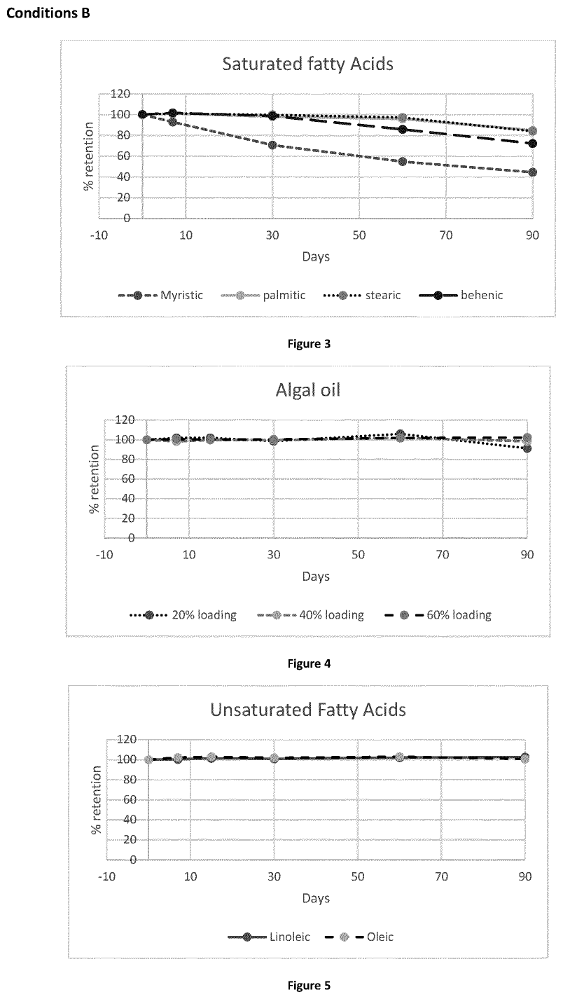

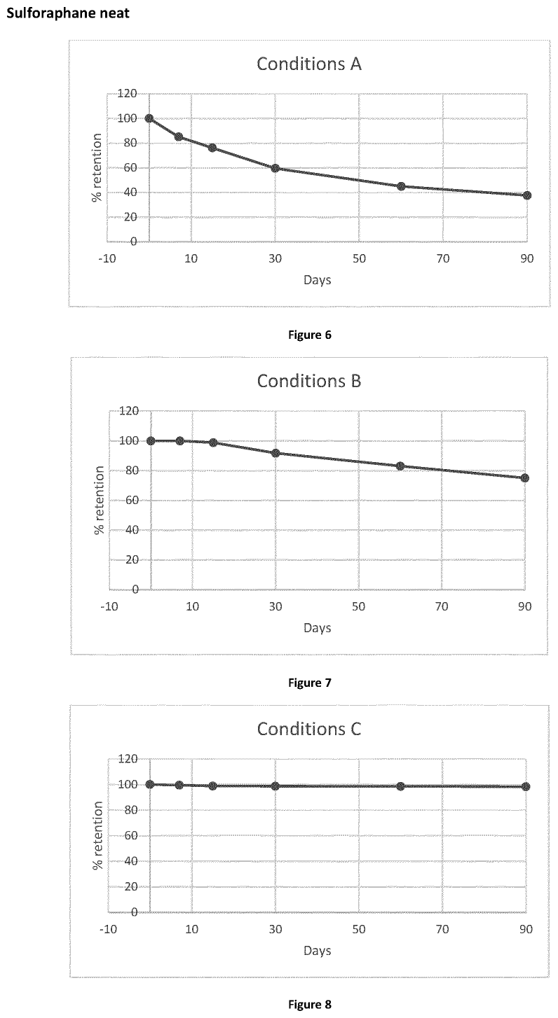

EP2120969A1 suggests using cyclodextrins to achieve sulforaphane stabilization. The theoretical loading of the sulforaphane-cyclodextrin complexes prepared therein ranges from 0.94 to 13.3% (see Table 1). Larger scale experiment resulted in even lower loading values (see Table 2). As evident by FIG. 3, the most stabilizing complex (alphaB) retained about 85% of the initial loading after 100 days at room temperature. The suggested complex however exhibits low loading values.

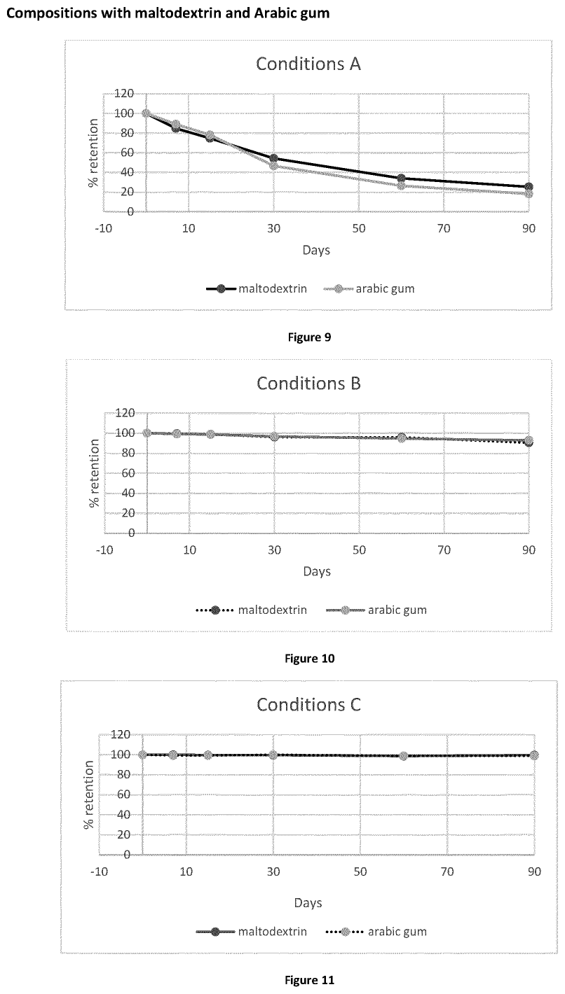

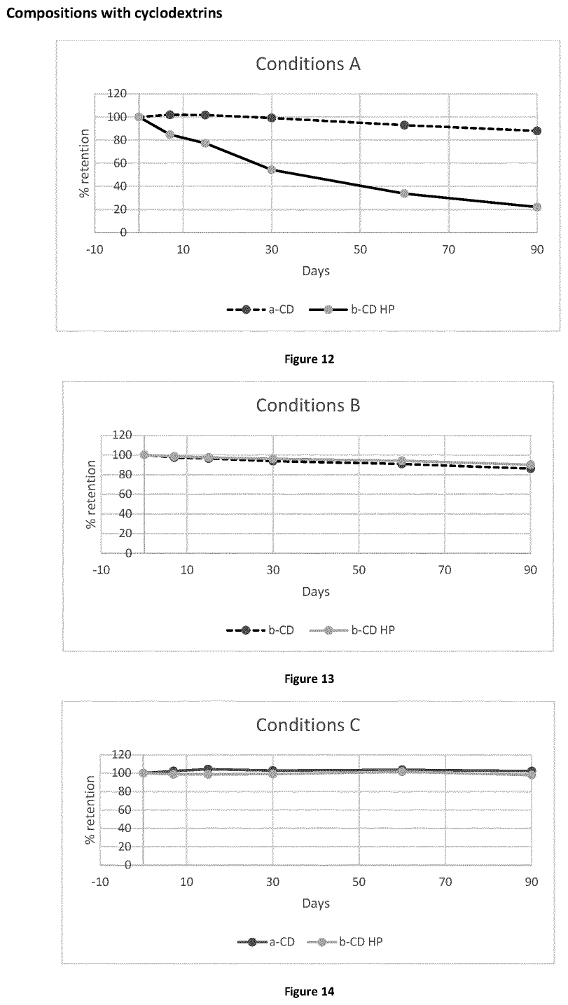

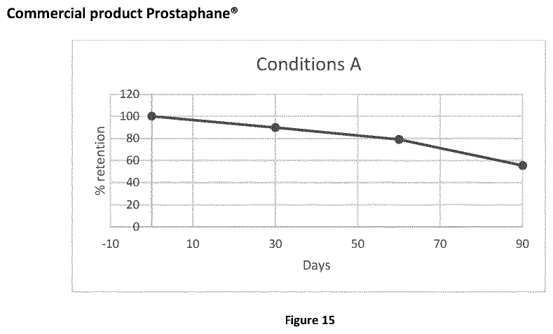

U.S. Pat. No. 9,254,331B2 is concerned with the provision of stable galenic compositions. The document emphasizes that, contrary to what has been reported, it is not light or oxidants that cause sulforaphane to degrade, but its capability of reacting with nucleophilic agents (column 5, In. 30-36). The proposed solution is the provision of galenic compositions comprising sulforaphane in an amount ranging from 0.01 to 15% and at least one anhydrous ester, the main chain and/or optionally the branched chains of which are free from and/or reactive groups or functions in an amount ranging from 0.1 to 99.9% by weight, based on the total weight of the cosmetically and pharmaceutically acceptable composition. According to this disclosure, the absence of nucleophilic groups in the above compositions contributes to the stability of sulforaphane. The document provides, among others, comparative examples in various other media. Notably, a 1% solution of sulforaphane in jojoba oil failed to maintain residual sulforaphane in acceptable levels (FIG. 5).

US2011003747A is concerned with the treatment of Epidermolysis bullosa simplex. The compositions prepared for topical treatment (e.g. example 3) comprise of sulforaphane and jojoba oil. However, the document is silent regarding the stability of those compositions.

US2006127996 is concerned with methods of direct extraction of isothiocyanates into the oil from glucosinolate-containing plants. Apparently, the extraction of sulforaphane from natural sources is difficult, due to the instability of sulforaphane. The method disclosed therein transfers sulforaphane quickly into the oil phase created by the parallel extraction of the vegetable oil ([0012]). This transfer is allegedly responsible for the stability of the isothiocyanates, as they are separated from the proteins present in the aqueous phase. Example 2C discloses a composition of about 5.2% w/w g sulforaphane/g broccoli seed oil. The document is silent regarding long-term stability of such compositions, it discloses only the stability of the sulforaphane during the extraction process. Furthermore, there is no disclosure about the chemical composition of the broccoli seed oil. It is noted that broccoli seed oil contains about 50% of erucic acid, a monounsaturated omega-9 fatty acid, denoted 22:1ω9 (source: www.circulating-oils-library.com). Erucic acid is not desirable in significant amounts in edible products, as it has been shown to cause heart lesions.

US2009081138A is concerned with an extraction method that produces an extraction meal comprising sulforaphane and low content of natural oils. According to this document erucic acid, contained in some natural oil plants, has been shown to cause heart lesions and therefore extraction meals containing significant amounts of erucic acid are unsuitable for use as food additives [0003]. The document is silent regarding stability. The extracts of the alleged invention are disclosed as having sulforaphane in amounts of 2.0 to 5.5% wt (see [0029]), however there is no indication in the examples whatsoever.

WO2019148250A1 is concerned with methods to protect bioactive compounds. Example 20 allegedly demonstrates that freeze dried broccoli aqueous suspensions and emulsions stored at −18° C. are stabilized by the presence of oil (tuna oil, DHA canola oil, canola oil, see example 6). No experimental data is provided and there is no kind of measurement of sulforaphane anywhere in the application as filed. No actual calculations can be done regarding the concentration of sulforaphane in said suspensions and emulsions, as the origin of sulforaphane is from a natural source of undetermined sulforaphane content. Furthermore, it is noted that neat sulforaphane is stable under deep freeze conditions (see Comparative Example 3).

EP2163238A2 discloses combination of sulforaphane with dicarboxylic acids for the preparation of cosmetic or dermatological composition for lightening the skin. The cosmetic or dermatological compositions comprise further ingredients. The document is silent on the stability of its preparations.

It would be desirable to provide compositions that stabilize sulforaphane, which is present in relatively high concentrations, to be used as an ingredient in the provision of further products, of pharmaceutical, nutraceutical, cosmetical or any other use. Furthermore, it would be even more desirable to provide stabilizing compositions of sulforaphane, wherein the other ingredients present are non-toxic or even more preferable, wherein the other ingredients are beneficial for dietary or other health related purposes, such as e.g. omega-3 or omega-6 fatty acids.

### SUMMARY OF THE INVENTION

As it is already known in literature (WO2012010644 and J. Agric. Food Chem. 1999, 47, 3121-3123) the major degradation impurity of Sulforaphane is its thiourea derivative, which appears to be the derivative that predominates in the extract derived from Broccoli. The formation of this impurity occurs as a result of isothiocyanate hydrolysis which generates the corresponding amine.

It is an object of the present invention to provide a composition which comprises sulforaphane in high concentration, suitable for use in preparing further compositions and products, which is chemically stable.

It is a further object of the present invention to provide a method for stabilizing sulforaphane during long-term storage.

The inventors have surprisingly found that fatty acids may be used to prepare sulforaphane compositions to stabilize the latter, such that it facilitates long term storage.

Advantageously the compositions disclosed in the present invention may be used commercially either alone or as a component in the compositions of pharmaceutical, food or drink products, nutraceutical products, dietary supplements, food additives, cosmetic, skin or hair products, and agricultural products.

### Definitions

A composition is defined as “stabilized”, when sulforaphane in said composition degrades less or more slowly than it does on its own, under the same conditions.

The fatty acids are defined according to the IUPAC Golden Book as “Aliphatic monocarboxylic acids derived from or contained in esterified form in an animal or vegetable fat, oil or wax”. In addition, the IUPAC Golden Book clarifies “Natural fatty acids commonly have a chain of 4 to 28 carbons (usually unbranched and even-numbered), which may be saturated or unsaturated. By extension, the term is sometimes used to embrace all acyclic aliphatic carboxylic acids”. Accordingly, the fatty acids employed in the present invention are not in esterified form, i.e. they are “free” fatty acids.

Fatty acids may be saturated or unsaturated, as disclosed above. Saturated fatty acids include but are not limited to Butyric acid, Valeric acid, Caproic acid, Enanthic acid, Caprylic acid, Pelargonic acid, Capric acid, Undecylic acid, Lauric acid, Tridecylic acid, Myristic acid, Pentadecylic acid, Palmitic acid, Margaric acid, Stearic acid, Nonadecylic acid, Arachidic acid, Heneicosylic acid, Behenic acid, Tricosylic acid, Lignoceric acid, Pentacosylic acid, Cerotic acid, Carboceric acid, Montanic acid, Nonacosylic acid, Melissic acid, Hentriacontylic acid, Lacceroic acid, Psyllic acid, Geddic acid, Ceroplastic acid, Hexatriacontylic acid, Heptatriacontylic acid, Octatriacontylic acid, Nonatriacontylic acid, Tetracontylic acid. Unsaturated fatty acids include but are not limited to Palmitoleic acid, cis-Vaccenic acid, Linoleic acid, Linoelaidic acid, γ-Linolenic acid, Oleic acid, Elaidic acid, cis-9-eicosenoic acid, cis-11-eicosenoic acid, Mead acid, Hexadecatrienoic acid, α-Linolenic acid (ALA), Stearidonic acid, Eicosadienoic acid, Eicosatrienoic acid, Eisocatetraenoic acid, Eicosapentaenoic acid (EPA), Heneicosapentaenoic acid, Docosapentaenoic acid (Clupanodonic acid), Docosahexaenoic acid (DHA), Tetracosapentaenoic acid, Tetracosahexaenoic acid, Dihomo-γ-linolenic acid, Arachidonic acid, Paullinic acid, Gondoic acid, Mead acid, Cervonic acid, Docosadienoic acid, Adrenic acid, Osbond acid (Docosapentaenoic acid), Tetracosatetraenoic acid, Tetracosapentaenoic acid, Docosatetraenoic acid, Erucic acid, Nervonic acid. Certain unsaturated fatty acids may be also categorized as polyunsaturated fatty acids, i.e. having more than one unsaturated bonds. Certain unsaturated fatty acids may be also categorized as omega-3, omega-6 or omega-9 fatty acids. Non-limiting examples of omega-3 fatty acids are Hexadecatrienoic acid, α-Linolenic acid, Stearidonic acid, Eicosatrienoic acid, Eicosatetraenoic acid, Eicosapentaenoic acid, Heneicosapentaenoic acid, Docosapentaenoic acid (Clupanodonic acid), Docosahexaenoic acid, Tetracosapentaenoic acid, Tetracosahexaenoic acid. Non-limiting examples of omega-6 fatty acids are Linoleic acid, γ-Linolenic acid, Eicosadienoic acid, Dihomo-γ-linolenic acid, Arachidonic acid, Docosadienoic acid, Adrenic acid, Docosapentaenoic acid (Osbond acid), Tetracosatetraenoic acid, Tetracosapentaenoic acid. Non-limiting examples of omega-9 fatty acids are Oleic acid, Eicosenoic acid, Mead acid, Erucic acid, Nervonic acid.

A dietary supplement is a composition consumed in order to supplement the diet for a nutritional purpose. Dietary supplements may take a variety of forms including but not limited to pills, capsules, tablets and liquid forms.

A food additive is a composition intended to be added to a food product in order to enhance the nutritional value of a diet that includes said food product.

The term “nutraceutical composition” or “functional food” is understood to mean a composition based on a food or foods possessing a positive and significant effect on health and on the prevention of pathologies.

The term “pharmaceutical composition” is understood to mean a physical mixture containing a therapeutic compound to be administered to a mammal, e.g., a human in order to prevent, treat or control a particular disease or condition affecting the mammal.

The term “cosmetic composition” is understood to mean compositions which are used to treat, care for or improve the appearance of the skin and/or the scalp.

### DETAILED DESCRIPTION OF THE INVENTION

During stability studies, it was observed that Sulforaphane degrades when left on bench even after a period of 7 days, affording a major impurity with retention time about 15.35 min and RRT 0.76 (relative retention time). The same impurity was also observed during a force degradation trial when diluting Sulforaphane in water and heated at 80° C. for 24 hrs. The HPLC analysis showed that this impurity was detected at more than 93% (chromatographic purity) in the sample.

As it is already known in literature (WO2012010644 and J. Agric. Food Chem. 1999, 47, 3121-3123) the major degradation impurity of Sulforaphane is its thiourea derivative. It was assumed that the impurity detected at 0.76 RRT is the thiourea derivative. Below is a possible pathway for its formation:

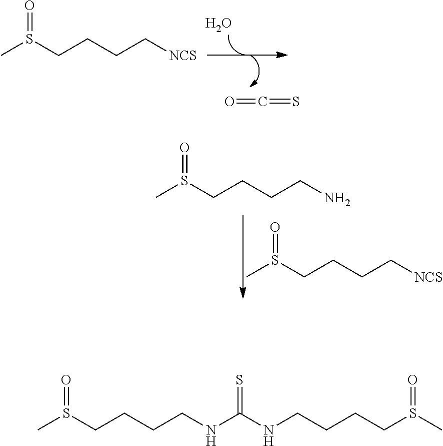

In order to determine the structure of the major degradation impurity found in the present study (RRT 0.76) a sample of synthetically produced Sulforaphane was left on bench for 6 months. HPLC analysis showed that the percentage of this impurity is about 74.8%.

This compound was further analyzed by means of LC-MS, ¹H and ¹³C-NMR. The data collected are shown below.

LC-MS: 312.85 (M⁺), 335.05 [(M+Na)⁺]

¹H-NMR (500 MHz, CDCl₃) 1.73-1.92 (m, 8H), 2.57 (s, 6H), 2.70-2.74 (m, 4H), 3.54-3.59 (m, 4H)

¹³C-NMR (125 MHz, CDCl₃) 19.9, 28.9, 38.6, 44.6, 53.5

All data given above coincide with those in literature (WO2012010644 example 9) and prove that the degradation impurity (RRT 0.76) is the thiourea derivative.

In a first embodiment, the present invention provides a composition comprising sulforaphane and one or more fatty acids, wherein sulforaphane is present in the composition in a concentration which is at least 10% w/w and said fatty acid or fatty acids are present in the composition in a concentration which is at least 1% w/w.

The inventors have surprisingly found that sulforaphane is stabilized in the presence of fatty acids. Compositions prepared by mixing sulforaphane with certain amounts of a fatty acid or a mixture of fatty acids exhibit a stabilizing effect with respect to residual sulforaphane. It was found that such compositions can maintain sulforaphane, as expressed by % w/w, more effectively, i.e. stabilize it, compared to neat sulforaphane, under certain storage conditions. This facilitates the distribution of sulforaphane as a component to be used for industrial purposes, for example in the preparation of various products, dietary supplements, food additives, pharmaceuticals, nutraceuticals, cosmetics etc, because it makes long-term storage easier and efficient. No complex techniques are required for the stabilization, such as encapsulation or complexation, neither extremely low temperatures.

The storage conditions studied in the present invention are of three types:

### Conditions A: 20-25° C.

### Conditions B: 2-8° C.

### Conditions C: −15° C.

In all three above types of conditions, the storage media is a simple dark glass vial or round-bottomed flask. Each sample is blown with Argon before securing the cap. No further treatment regarding the atmosphere is performed. The vial is capped and secured by use of a commercially available appropriate means for sealing the cap, e.g. Parafilm. The storage media is not restrictive with respect to the present invention. Any means of storage may be used, as long as it does not interact with its content and can provide or allow for light protection similar to that of Amber or dark glass.

The fatty acid or fatty acids used in the compositions of the present invention may be any kind of fatty acids. The compositions of the present invention may be prepared by using either mixtures of fatty acids, or a single type of fatty acid.

In a preferred embodiment, only a single type of fatty acid is used.

The fatty acids may be of synthetic or natural source.

In a preferred embodiment the natural source of fatty acids are natural oils. Suitable natural oils comprise fatty acid or fatty acids in an amount such that after employing it into the compositions of the present invention, the concentration of the fatty acid or fatty acids in the composition is as provided in the various embodiments.

Examples of natural oils include but are not limited to tuna oil, herring oil, mackerel oil, sardine oil, cod liver oil, menhaden oil, shark oil, algal oil, squid oil, squid liver oil, krill oil, canola oil, sunflower oil, avocado oil, soya oil, borage oil, evening primrose oil, safflower oil, flaxseed oil, olive oil, pumpkinseed oil, hemp seed oil, wheat germ oil, palm oil, palm olein, palm kernel oil, coconut oil, grapeseed oil.

In a preferred embodiment the fatty acid or fatty acids are Lauric acid, Myristic acid, Palmitic acid, Stearic acid, Arachidic acid, Behenic acid, Palmitoleic acid, cis-Vaccenic acid, Linoleic acid, γ-Linolenic acid, Oleic acid, cis-9-eicosenoic acid, cis-11-eicosenoic acid, α-Linolenic acid (ALA), Stearidonic acid, Eisocatetraenoic acid, Eicosapentaenoic acid (EPA), Docosapentaenoic acid (Clupanodonic acid), Docosahexaenoic acid (DHA), Dihomo-γ-linolenic acid, Arachidonic acid and Nervonic acid.

In a more preferred embodiment the fatty acid or fatty acids are Palmitic acid, Stearic acid, Arachidic acid, Palmitoleic acid, Linoleic acid, γ-Linolenic acid, Oleic acid, α-Linolenic acid (ALA), Eicosapentaenoic acid (EPA), Docosahexaenoic acid (DHA), Dihomo-γ-linolenic acid and Arachidonic acid. In an even more preferred embodiment, the fatty acid or fatty acids are Palmitoleic acid, Linoleic acid, γ-Linolenic acid, Oleic acid, α-Linolenic acid (ALA), Eicosapentaenoic acid (EPA), Docosahexaenoic acid (DHA), Dihomo-γ-linolenic acid and Arachidonic acid.

In a preferred embodiment the fatty acid or fatty acids have at least 16 carbon atoms in the alkyl chain.

In an even more preferred embodiment, the fatty acid or acids have from 16 to 22 carbon atoms in the alkyl chain.

In a preferred embodiment at least one of the fatty acid or fatty acids are selected from omega-3 fatty acids or omega-6 fatty acids or omega-9 fatty acids.

In a preferred embodiment, the fatty acid or fatty acids used in the disclosed compositions are fatty acids which are not toxic to humans or animals.

The provided compositions may be in liquid or solid phase when under storage conditions. Some fatty acids, for instance, are liquid in room temperature but solidify at lower temperatures. This is not restrictive with respect to the present invention.

Sulforaphane may be of natural or synthetic origin. Sulforaphane of natural origin is extracted from plants, e.g. cruciferous vegetables such as broccoli. Such natural extracts usually contain high amounts of erucic acid, a fatty acid which is found in abundance in broccoli seeds.

In a preferred embodiment sulforaphane is of synthetic origin and thus it is not part of a natural extract. The stabilized compositions prepared from synthetic sulforaphane thus advantageously have a fully characterized profile of ingredients. Said compositions may also be free of other impurities or minor ingredients, such as carbohydrates, proteins, lipids, fatty acids such as erucic acid and other natural ingredients present in cruciferous plants, from which sulforaphane is usually extracted.

In a preferred embodiment the provided compositions comprise erucic acid in a concentration of not more than 5% w/w. More preferably, the provided compositions comprise erucic acid in a concentration of not more than 2% w/w. Still more preferably, the provided compositions comprise erucic acid in a concentration of not more than 1% w/w. Still more preferably, the provided compositions comprise erucic acid in a concentration of not more than 0.5% w/w. Even more preferably, the provided compositions comprise erucic acid in a concentration of not more than 0.1% w/w.

In an embodiment, the sulforaphane concentration in the provided compositions is at least 10% w/w and the fatty acid or fatty acids concentration in the provided compositions is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In an ever more preferred embodiment, the fatty acid or fatty acids are present in a concentration which is at least 40% w/w. In a still more preferred embodiment, the fatty acid or fatty acids are present in a concentration which is at least 50% w/w. In an even more preferred embodiment, the fatty acid or fatty acids are present in a concentration which is at least 60% w/w. In a still more preferred embodiment, the fatty acid or fatty acids are present in a concentration which is at least 70% w/w.

In a preferred embodiment, the sulforaphane concentration in the provided compositions is at least 15% w/w and the fatty acid or fatty acids concentration in the provided compositions is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In an ever more preferred embodiment, the fatty acid or fatty acids are present in a concentration which is at least 40% w/w. In a still more preferred embodiment, the fatty acid or fatty acids are present in a concentration which is at least 50% w/w. In an even more preferred embodiment, the fatty acid or fatty acids are present in a concentration which is at least 60% w/w. In a still more preferred embodiment, the fatty acid or fatty acids are present in a concentration which is at least 70% w/w.

In another preferred embodiment, the sulforaphane concentration in the provided compositions is at least 20% w/w and the fatty acid or fatty acids concentration in the provided compositions is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In an ever more preferred embodiment, the fatty acid or fatty acids are present in a concentration which is at least 40% w/w. In a still more preferred embodiment, the fatty acid or fatty acids are present in a concentration which is at least 50% w/w. In an even more preferred embodiment, the fatty acid or fatty acids are present in a concentration which is at least 60% w/w. In a still more preferred embodiment, the fatty acid or fatty acids are present in a concentration which is at least 70% w/w.

In another preferred embodiment, the sulforaphane concentration in the provided compositions is at least 30% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In an ever more preferred embodiment, the fatty acid or fatty acids are present in a concentration which is at least 40% w/w. In a still more preferred embodiment, the fatty acid or fatty acids are present in a concentration which is at least 50% w/w. In an even more preferred embodiment, the fatty acid or fatty acids are present in a concentration which is at least 60% w/w.

In another preferred embodiment, the sulforaphane concentration in the provided compositions is at least 40% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In an ever more preferred embodiment, the fatty acid or fatty acids are present in a concentration which is at least 40% w/w. In a still more preferred embodiment, the fatty acid or fatty acids are present in a concentration which is at least 50% w/w.

In another preferred embodiment, the sulforaphane concentration in the provided compositions is at least 50% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w. In another preferred embodiment, the fatty acid or fatty acids are present in a concentration which is at least 40% w/w.

In another preferred embodiment, the sulforaphane concentration in the provided compositions is at least 60% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w. In a yet more preferred embodiment, the fatty acid or fatty acids are present in a concentration which is at least 30% w/w.

In another preferred embodiment, the sulforaphane concentration in the provided compositions is at least 70% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w. In a still more preferred embodiment, the fatty acid or fatty acids are present in a concentration which is at least 20% w/w.

In another preferred embodiment, the sulforaphane concentration in the provided compositions is at least 80% w/w and the fatty acid or fatty acids are present in the composition in a concentration which is at least 1% w/w. In a more preferred embodiment, the fatty acid or fatty acids are present in a concentration which is at least 5% w/w. In another preferred embodiment, the fatty acid or fatty acids are present in a concentration which is at least 10% w/w. In another preferred embodiment, the fatty acid or fatty acids are present in a concentration which is at least 15% w/w.

In another preferred embodiment, the sum of the concentrations of sulforaphane and fatty acid or fatty acids in any of the above embodiments is at least 70% w/w. In a still more preferred embodiment, the sum of the concentrations of sulforaphane and fatty acid or fatty acids is at least 80% w/w. In a still more preferred embodiment, the sum of the concentrations of sulforaphane and fatty acid or fatty acids is at least 90% w/w.

In a preferred embodiment, the present invention provides a composition consisting of sulforaphane and one or more fatty acids, wherein sulforaphane is present in the composition in a concentration which is at least 10% w/w.

In this embodiment all features of the compositions are as described above, with the additional feature that the compositions consist of sulforaphane and a fatty acid or fatty acids. This implicitly means that said fatty acid or fatty acids are present in this embodiment's compositions at concentration which, when added to sulforaphane concentration, sums up to 100% w/w.

In a preferred embodiment, there is provided a composition consisting of sulforaphane and one or more fatty acids, wherein sulforaphane is present in the composition in a concentration which is at least 20% w/w. In a yet more preferred embodiment, sulforaphane concentration is at least 30% w/w, more preferably 40% w/w, even more preferably 50% w/w, still more preferably 60% w/w, more preferably 70% w/w, still more preferably 80% w/w. In all those embodiments it is implicitly meant that the remaining amount % w/w required to reach 100 is made up from fatty acid or fatty acids.

The compositions provided in this embodiment provide sulforaphane in a stabilized composition, which is made up of very few components. This makes said compositions more versatile in their potential use at the nutraceutical, pharmaceutical or cosmetic industry. By appropriate selection of the fatty acid or fatty acids, it is also possible to stabilize sulforaphane in a composition such that it allows direct use in the preparation or manufacturing of other products. Furthermore, the compositions provided in the present invention stabilize sulforaphane in temperatures higher than the temperature required to preserve neat sulforaphane. In other words, sulforaphane can be retained in the disclosed compositions and stored at temperatures that are feasible for the purpose of storing materials to be used in manufacturing of other goods or for commercial purposes.

In a further embodiment of the present invention there is provided a method for stabilizing sulforaphane by providing a composition as described in the previous embodiments.

In a further embodiment of the present invention there is provided a use of the compositions as disclosed herein in preparation of a nutraceutical, veterinary, cosmetic or pharmaceutical composition.

### Examples

### Starting Materials

Sulforaphane may be prepared synthetically according to procedures available in the prior art, for example according to ChemBioChem 2008, 9, p. 729; Mays et al Identification, synthesis, and enzymology of non-natural glucosinolate chemopreventive candidates”.

Fatty acids and natural oils were obtained from commercial sources or prepared according to procedures available in the prior art, as reviewed in https://doi.org/10.1016/j.egypro.2017.03.1137. Dextrins and natural gums used in the comparative examples were purchased from commercial sources. Prostaphane® composition was purchased from retail suppliers.

The algal oil employed in the experiments is comprised of free fatty acids. The content of the algal oil with respect to fatty acids is defined by GC method by derivatization of the free fatty acids into their ethyl esters. The samples were analyzed in a GC-FID instrument with a DB-23 fused silica column (60 m x 0.25 mmid, 0.25 μm). Erucic acid was not detected with the GC method in algal oil samples.

| Fatty acid | Area |
|---|---|
| (as ethyl ester) | % (GC) |
| Palmitic Acid | 12,58 |
| Palmitoleic Acid | 18,75 |
| Arachidonic Acid | 4,09 |
| Eicosapentaenoic Acid | 34,61 |

### Analysis Overview of Stability Studies

The Sulforaphane formulations were analyzed with HPLC (PDA/UV at 240 nm) using standards for identification and quantification purpose.

### Mobile Phase A/B

Take 1 mL of H₃PO₄ into a 1 L volumetric flask containing about 500 mL of water, dilute to the volume with water and mix well/Acetonitrile.

### Chromatographic Conditions

| | HPLC equipped with | |
|---|---|---|
| Instrument | PDA or UV detector | |
| Column Stationary phase | ACE 5 C18-PFP (250 × 4.6 | |
| | mm i.d., 5 μm) | |
| Flow rate | 1.0 | mL/min |
| Column oven temperature | 15° | C. |
| Autosampler temperature | 5° | C. |
| Detector | PDA or UV | |
| Detector wavelength | 240 | nm |
| Run time | 60 | min |
| Injection volume | 20 | μL |

| | |
|---|---|
| Elution | Gradient |

### Sample Preparation

Transfer accurately about 20 mg of sample in to a 20 mL clean and dry volumetric flask. Dilute to the volume with diluent.

Sulphoraphane retention time is about 20 mins

In the below examples of preparing the compositions of the present invention the temperature storing conditions are indicated as follows:

Conditions A: bench (20-25° C.)
Conditions B: fridge, 2-8° C.
Conditions C: fridge-freezer, (−15) ° C.

### Example 1: Stabilization of Sulforaphane in a Single Fatty Acid

General procedure: In a dark glass round bottom flask add 200 mg of sulforaphane in 20 ml ethanol and 600 mg of fatty acid. Stir for 1 hour and evaporate solvents to dryness. Blow with Argon, cap, apply Parafilm and store under conditions A or B.

### Example 1a: Myristic Acid (1-Tetradecanoic Acid, C14:0)

### Conditions B:

| | zero time | 7 days | 1 month | 2 months | 3 months |
|---|---|---|---|---|---|
| RRT 0,76 | 0,65 | 0,70 | 0,73 | 0,67 | 0,70 |
| RRT 0,96 | 3,37 | 11,08 | 32,30 | 47,30 | 55,82 |
| Sulforaphane | 88,38 | 82,00 | 62,39 | 48,39 | 39,19 |
| Sulfone | 1,61 | 1,41 | 1,1 | | 0,69 |

### Example 1b: Palmitic Acid (Hexadecenoic Acid, C16:0)

### Conditions B:

| | zero time | 7 days | 1 month | 2 months | 3 months |
|---|---|---|---|---|---|
| RRT 0,76 | 0,73 | 0,81 | 1,20 | 1,22 | 4,86 |
| RRT 0,96 | 2,81 | 2,69 | 3,46 | 4,12 | 4,16 |
| Sulforaphane | 88,41 | 89,36 | 86,64 | 84,81 | 74,98 |
| Sulfone | 1,61 | 1,64 | 1,66 | 1,23 | 1,35 |

### Example 1c: Stearic Acid (Octadecanoic Acid, C18:0)

### Conditions B:

| | zero time | 7 days | 1 month | 2 months | 3 months |
|---|---|---|---|---|---|
| RRT 0,76 | 0,91 | 0,957 | 1,61 | 5,25 | 9,55 |
| RRT 0,96 | 2,05 | 1,98 | 2,40 | 2,01 | 2,32 |
| Sulforaphane | 88,89 | 89,79 | 88,64 | 86,41 | 74,62 |
| Sulfone | 1,51 | 1,71 | 1,61 | | 1,33 |

### Example 1d: Behenic Acid (Docosanoic Acid, C22:0)

### Conditions B:

| | zero time | 7 days | 1 month | 2 months | 3 months |
|---|---|---|---|---|---|
| RRT 0,76 | 1,14 | 0,89 | 1,26 | 6,64 | 12,16 |
| RRT 0,96 | 3,39 | 3,26 | 3,66 | 3,85 | 3,66 |
| Sulforaphane | 87,98 | 89,41 | 86,75 | 75,48 | 63,47 |
| Sulfone | 1,63 | 1,69 | 1,56 | | 1,14 |

### Example 1e: Oleic Acid [(9Z)-Octadec-9-Enoic Acid, 18:1 Cis-9]

### Conditions A:

| | zero | 7 | 15 | 1 | 2 | 3 |
|---|---|---|---|---|---|---|
| | time | days | days | month | months | months |
| RRT 0.76 | 0.62 | 0.52 | 1.14 | 4.06 | 16.96 | 31.60 |
| RRT 0.96 | 1.01 | 1.08 | 1.12 | 1.34 | 1.31 | 1.00 |
| Sulforaphane | 91.51 | 93.02 | 92.73 | 87.98 | 71.12 | 44.07 |
| Sulfone | 1.69 | 1.74 | 1.80 | 1.38 | 1.42 | 0.83 |

### Conditions B:

| | zero | 7 | 15 | 1 | 2 | 3 |
|---|---|---|---|---|---|---|
| | time | days | days | month | months | months |
| RRT 0.76 | 0.62 | 0.64 | 0.22 | 0.40 | 0.46 | 1.26 |
| RRT 0.96 | 1.01 | 1 | 1.04 | 1.09 | 1.12 | 1.17 |
| Sulforaphane | 91.51 | 93.49 | 94.18 | 93.23 | 94.25 | 92.09 |
| Sulfone | 1.69 | 1.72 | 1.78 | 1.71 | 1.79 | 1.76 |

### Example 1f: Linoleic acid [(9Z,12Z)-octadeca-9,12-dienoic acid, 18:2 cis-9,12]

### Conditions A:

| | zero | 7 | 15 | 1 | 2 | 3 |
|---|---|---|---|---|---|---|
| | time | days | days | month | months | months |
| RRT 0.76 | 0.65 | 1.43 | 1.88 | 4.01 | 13.93 | 14.21 |
| RRT 0.96 | 1.03 | 1.11 | 1.23 | 1.43 | 1.44 | 1.43 |
| Sulforaphane | 93.14 | 94.04 | 93.84 | 90.30 | 76.89 | 77.28 |
| Sulfone | 1.69 | 1.69 | 1.76 | 1.71 | 1.50 | 3.37 |

### Conditions B:

| | zero | 7 | 15 | 1 | 2 | 3 |
|---|---|---|---|---|---|---|
| | time | days | days | month | months | months |
| RRT 0.76 | 0.65 | 0.76 | 0.51 | 0.98 | 0.80 | 0.83 |
| RRT 0.96 | 1.03 | 1.02 | 1.04 | 1.06 | 1.10 | 0.95 |
| Sulforaphane | 93.14 | 93.5 | 94.34 | 93.86 | 95.11 | 95.59 |
| Sulfone | 1.69 | 1.68 | 1.76 | 1.69 | 1.76 | 1.82 |

### Example 2: Stabilization of Sulforaphane in Algal Oil

General procedure: In a dark glass vial mix the appropriate amount of sulforaphane and algal oil to prepare a mixture of total weight 1.0 g. Blow with Argon, cap, apply parafilm and store under conditions A or B.

### Example 2a: Algal Oil, Sulforaphane Loading 20% w/w

### Conditions A:

| | zero | 7 | 15 | 1 | 2 | 3 |
|---|---|---|---|---|---|---|
| | time | days | days | month | months | months |
| RRT 0.76 | 0.01 | 0.04 | 0.18 | 2.36 | 12.44 | 14.96 |
| RRT 0.96 | 0.93 | 0.25 | | | 0.50 | |
| Sulforaphane | 91.80 | 83.29 | 95.12 | 89.13 | 52.78 | 56.61 |
| Sulfone | | 1.42 | 2.48 | 2.70 | 1.53 | 1.41 |

### Conditions B:

| | zero | 7 | 15 | 1 | 2 | 3 |
|---|---|---|---|---|---|---|
| | time | days | days | month | months | months |
| RRT 0.76 | 0.01 | 0.04 | 0.02 | 1.46 | 0.06 | 1.20 |
| RRT 0.96 | 0.93 | 0.70 | 0.44 | 0.95 | | |
| Sulforaphane | 91.80 | 92.80 | 92.75 | 89.82 | 96.50 | 83.09 |
| Sulfone | 2.06 | 1.36 | 1.98 | 2.13 | 2.02 | 3.03 |

### Example 2b: Algal Oil, Sulforaphane Loading 40% w/w

### Conditions A:

| | zero | 7 | 15 | 1 | 2 | 3 |
|---|---|---|---|---|---|---|
| | time | days | days | month | months | months |
| RRT 0.76 | 0.11 | 0.05 | 0.06 | 1.49 | 8.25 | 14.77 |
| RRT 0.96 | 0.94 | 0.80 | 0.82 | 1.92 | 1.23 | 1.09 |
| Sulforaphane | 94.06 | 92.69 | 95.15 | 89.93 | 74.71 | 57.95 |
| Sulfone | 1.83 | 1.42 | 1.45 | 1.98 | 1.96 | 1.48 |

### Conditions B:

| | zero | 7 | 15 | 1 | 2 | 3 |
|---|---|---|---|---|---|---|
| | time | days | days | month | months | months |
| RRT 0.76 | 0.11 | 0.06 | 0.01 | | | |
| RRT 0.96 | 0.94 | 0.87 | 0.73 | | 0.68 | 0.42 |
| Sulforaphane | 94.06 | 92.4 | 93.78 | 93.76 | 95.79 | 92.52 |
| Sulfone | 1.83 | 1.55 | 1.85 | 2.27 | 1.78 | 2.24 |

### Example 2c: Algal Oil, Sulforaphane Loading 60% w/w

### Conditions A:

| | zero | 7 | 15 | 1 | 2 | 3 |
|---|---|---|---|---|---|---|
| | time | days | days | month | months | months |
| RRT 0.76 | 0.21 | 0.02 | 0.03 | 0.19 | 9.1 | 15.37 |
| RRT 0.96 | 0.91 | 1.19 | 1.41 | | 2.72 | 2.81 |
| Sulforaphane | 92.81 | 93.70 | 93.96 | 89.77 | 78.01 | 68.51 |
| Sulfone | 1.73 | 1.67 | 1.79 | 1.89 | 1.77 | 1.52 |

### Conditions B:

| | zero | 7 | 15 | 1 | 2 | 3 |
|---|---|---|---|---|---|---|
| | time | days | days | month | months | months |
| RRT 0.76 | 0.21 | 0.42 | | 0.08 | 0.03 | 0.03 |
| RRT 0.96 | 0.91 | 0.94 | 0.94 | 1.00 | 1.15 | 1.32 |
| Sulforaphane | 92.81 | 93.47 | 92.94 | 93.03 | 94.34 | 94.83 |
| Sulfone | 1.73 | 1.72 | 1.73 | 1.85 | 1.99 | 2.12 |

### Comparative Example 3: Sulforaphane Neat

General procedure: Neat sulforaphane, prepared according to prior art procedures, was placed in a dark glass vial, blown with Argon, capped, secured with parafilm and stored under conditions A or C.

### Conditions A:

| | zero | 7 | 15 | 1 | 2 | 3 |
|---|---|---|---|---|---|---|
| | time | days | days | month | months | months |
| RRT 0.76 | 0.01 | 15.87 | 21.61 | 29.16 | 34.37 | 38.38 |
| RRT 0.96 | 0.15 | 2.62 | 3.20 | 3.88 | 3.95 | 4.11 |
| Sulforaphane | 94.74 | 65.48 | 53.28 | 36.35 | 24.51 | 19.98 |
| Sulfone | 3.40 | 2.32 | 1.92 | 1.31 | 0.84 | 0.78 |

### Conditions C:

| | zero | 7 | 15 | 1 | 2 | 3 |
|---|---|---|---|---|---|---|
| | time | days | days | month | months | months |
| RRT 0.76 | 0.01 | | 0.62 | 0.25 | 0.27 | 0.23 |
| RRT 0.96 | 0.15 | 0.15 | 0.03 | 0.23 | 0.25 | 0.51 |
| Sulforaphane | 94.74 | 95.21 | 97.23 | 94.27 | 94.43 | 94.51 |
| Sulfone | 3.40 | 3.38 | | 3.4 | 3.42 | 3.44 |

### Comparative Example 4: Sulforaphane with Alpha-Cyclodextrine

Preparation of sulforaphane and alpha-cyclodextrine complex was performed according to US2008176942A1 Formulation type C (theoretical load reported 4.73%). The prepared complex was placed in a dark glass vial, blown with Argon, capped, secured with parafilm and stored under conditions A or C.

### Conditions A:

| | zero | 7 | 15 | 1 | 2 | 3 |
|---|---|---|---|---|---|---|
| | time | days | days | month | months | months |
| RRT 0.76 | 0.15 | 1.19 | 2.00 | 4.08 | 6.5 | 7.83 |
| Sulforaphane | 93.62 | 95.36 | 95.21 | 92.76 | 86.9 | 82.32 |
| Sulfone | 2.11 | 2.23 | 2.19 | 2.1 | 2.1 | 1.87 |

### Conditions C:

| | zero | 7 | 15 | 1 | 2 | 3 |
|---|---|---|---|---|---|---|
| | time | days | days | month | months | months |
| RRT 0.76 | 0.15 | 0.14 | | | 0.22 | 0.281 |
| Sulforaphane | 93.62 | 95.72 | 97.62 | 96.27 | 96.9 | 95.7 |
| Sulfone | 2.11 | 2.18 | 2.26 | 1.95 | 2.35 | 2.16 |

### Comparative Example 5: Sulforaphane with Beta-Cyclodextrine

Preparation of sulforaphane and beta-cyclodextrine complex was performed according to US2008176942A1 Formulation type C (theoretical load reported 13.3%). The prepared complex was placed in a dark glass vial, blown with Argon, capped, secured with parafilm and stored under conditions B.

### Conditions B:

| | zero | 7 | 15 | 1 | 2 | 3 |
|---|---|---|---|---|---|---|
| | time | days | days | month | months | months |
| RRT 0.73 | 5.87 | 7.68 | 9.09 | 11.66 | 14.55 | 18.73 |
| RRT 0.76 | 0.11 | 0.14 | 0.17 | 0.10 | 0.17 | 0.26 |
| Sulforaphane | 89.45 | 87.049 | 86.19 | 83.7 | 81.13 | 76.94 |
| Sulfone | 3.92 | 3.8 | 3.80 | 3.70 | 3.52 | 3.44 |

### Comparative Example 6: Sulforaphane with Hydroxypropyl-Beta-Cyclodextrine

Preparation of sulforaphane and hydroxypropyl-beta-cyclodextrine complex was performed according to US2008176942A1 Formulation type C. The prepared complex was placed in a dark glass vial, blown with Argon, capped, secured with parafilm and stored under conditions A, B or C.

### Conditions A:

| | zero | 7 | 15 | 1 | 2 | 3 |
|---|---|---|---|---|---|---|
| | time | days | days | month | months | months |
| RRT 0.76 | 1.04 | 5.35 | 7.47 | 13.62 | 21.58 | 26.75 |
| RRT 0.77 | 1.89 | 8.85 | 11.40 | 33.84 | 27.68 | 33.52 |
| Sulforaphane | 92.25 | 78.21 | 71.34 | 50.02 | 31.08 | 20.39 |
| Sulfone | 3.31 | 2.76 | 2.56 | 1.80 | 1.09 | 0.73 |

### Conditions B:

| | zero | 7 | 15 | 1 | 2 | 3 |
|---|---|---|---|---|---|---|
| | time | days | days | month | months | months |
| RRT 0.76 | 1.04 | 1.36 | 1.63 | 1.83 | 2.18 | 3.54 |
| RRT 0.77 | 1.89 | 2.18 | 2.48 | 4.22 | 5.78 | 5.60 |
| Sulforaphane | 92.25 | 90.88 | 89.85 | 88.53 | 86.71 | 82.94 |
| Sulfone | 3.31 | 3.22 | 3.22 | 3.16 | 3.10 | 3.01 |

### Conditions C:

| | zero | 7 | 15 | 1 | 2 | 3 |
|---|---|---|---|---|---|---|
| | time | days | days | month | months | months |
| RRT 0.76 | 1.04 | 1.15 | 1.26 | 1.94 | | 1.58 |
| RRT 0.77 | 1.89 | 1.81 | 1.96 | 2.39 | 1.36 | 2.22 |
| Sulforaphane | 92.25 | 91.04 | 91.03 | 91.27 | 93.68 | 90.28 |
| Sulfone | 3.31 | 3.22 | 3.28 | 3.26 | 3.40 | 3.28 |

### Comparative Example 7: Sulforaphane with Maltodextrin

Preparation of composition: 1.0 g of sulforaphane is dissolved in 2.0 ml DM water. 5.0 g of Maltodextrin are added in 15 ml DM water under stirring followed by the sulforaphane solution. The mixture is stirred for 24 hours and then stored at −80° C. for another 24 hours. The mixture is then subjected to freeze drying. 1.69 g are collected, placed in dark glass vial, blown with Argon, capped, secured with parafilm and stored under conditions A, B or C.

### Conditions A:

| | zero | 7 | 15 | 1 | 2 | 3 |
|---|---|---|---|---|---|---|
| | time | days | days | month | months | months |
| RRT 0.76 | 0.24 | 6.95 | 11.95 | 24.59 | 36.25 | 43.00 |
| Sulforaphane | 95.09 | 80.55 | 71.13 | 51.59 | 32.33 | 24.06 |
| Sulfone | 3.41 | 2.84 | 2.55 | 1.87 | 1.16 | 0.88 |

### Conditions B:

| | zero | 7 | 15 | 1 | 2 | 3 |
|---|---|---|---|---|---|---|
| | time | days | days | month | months | months |
| RRT 0.76 | 0.24 | 0.45 | 0.71 | 1.51 | 2.87 | |
| Sulforaphane | 95.09 | 94.63 | 94.03 | 91.39 | 91.13 | 85.93 |
| Sulfone | 3.41 | 3.36 | 3.40 | 3.27 | 3.24 | 3.14 |

### Conditions C:

| | zero | 7 | 15 | 1 | 2 | 3 |
|---|---|---|---|---|---|---|
| | time | days | days | month | months | months |
| RRT 0.76 | 0.24 | 0.33 | 0.39 | 0.47 | 0.54 | 0.70 |
| Sulforaphane | 95.09 | 94.95 | 94.66 | 94.66 | 93.84 | 94.70 |
| Sulfone | 3.41 | 3.37 | 3.39 | 3.39 | 3.30 | 3.46 |

### Comparative Example 8: Sulforaphane with Arabic Gum

Preparation of composition: 1.0 g of sulforaphane is dissolved in 2.0 ml DM water. 5.0 g of Arabic gum are added in 15 DM water under stirring followed by the sulforaphane solution. The mixture is stirred for 24 hours and then stored at −80° C. for another 24 hours. The mixture is then subjected to freeze drying. 1.69 g are collected, placed in dark glass vial, blown with Argon, capped, secured with parafilm and stored under conditions A, B or C.

### Conditions A:

| | zero | 7 | 15 | 1 | 2 | 3 |
|---|---|---|---|---|---|---|
| | time | days | days | month | months | months |
| RRT 0.76 | 0.39 | 6.02 | 14.58 | 39.35 | 57.41 | 63.58 |
| Sulforaphane | 94.74 | 84.28 | 74.05 | 44.16 | 24.88 | 17.11 |
| Sulfone | 3.42 | 2.98 | 2.70 | 1.64 | 0.94 | 0.61 |

### Conditions B:

| | zero | 7 | 15 | 1 | 2 | 3 |
|---|---|---|---|---|---|---|
| | time | days | days | month | months | months |
| RRT 0.76 | 0.39 | 0.67 | 1.00 | 1.91 | 2.87 | 3.93 |
| Sulforaphane | 94.74 | 93.91 | 93.47 | 91.63 | 89.67 | 87.48 |
| Sulfone | 3.42 | 3.34 | 3.40 | 3.28 | 3.20 | 3.17 |

### Conditions C:

| | zero | 7 | 15 | 1 | 2 | 3 |
|---|---|---|---|---|---|---|
| | time | days | days | month | months | months |
| RRT 0.76 | 0.39 | 0.46 | 0.56 | 0.67 | 0.87 | |
| Sulforaphane | 94.74 | 94.11 | 94.11 | 94.71 | 93.65 | 93.71 |
| Sulfone | 3.42 | 3.33 | 3.40 | 3.41 | 3.36 | 3.40 |

### Comparative Example 9: Prostaphane®

Each tab contains 10 mg of sulphorafane. Tablets are grinded by mortar and pestle and the respective solution is prepared with diluent. Each stability measurement is performed by removing two tabs from the blister at the appropriate time station.

### Conditions A:

| | zero time | 1 month | 2 months | 3 months |
|---|---|---|---|---|
| RRT 0,75 | 15,55 | 30,12 | 41,15 | 37,29 |
| RRT 0,82 | 4,59 | 3,74 | 2,94 | 2,33 |
| RRT 0,86 | 1,36 | 0,91 | 0,75 | 0,69 |
| SLFN | 25,83 | 23,18 | 20,42 | 14,33 |

1. A composition comprising sulforaphane and one or more fatty acids, wherein sulforaphane is present in the composition in a concentration which is at least 10% w/w and said fatty acid or fatty acids are present in the composition in a concentration which is at least 1% w/w.

2. A composition according to claim 1, wherein sulforaphane is present in the composition in a concentration which is at least 15% w/w.

3. A composition according to claim 1, wherein sulforaphane is present in the composition in a concentration which is at least 20% w/w.

4. A composition according to claim 1, wherein sulforaphane is present in the composition in a concentration which is at least 30% w/w.

5. A composition, according to claims 1-4, wherein said fatty acid or fatty acids are present in the composition in a concentration which is at least 5% w/w.

6. A composition, according to claims 1-4, wherein said fatty acid or fatty acids are present in the composition in a concentration which is at least 10% w/w.

7. A composition, according to claims 1-4, wherein said fatty acid or fatty acids are present in the composition in a concentration which is at least 15% w/w.

8. A composition, according to claims 1-4, wherein said fatty acid or fatty acids are present in the composition in a concentration which is at least 20% w/w.

9. A composition, according to claims 1-4, wherein said fatty acid or fatty acids are present in the composition in a concentration which is at least 30% w/w.

10. A composition according to any preceding claim, wherein the sum of the concentrations of sulforaphane and fatty acid or fatty acids is at least 70%.

11. A composition consisting of sulforaphane and one or more fatty acids, wherein sulforaphane is present in the composition in a concentration which is at least 10%.

12. A composition according to claim 11, wherein sulforaphane is present in the composition in a concentration which is at least 15% w/w.

13. A composition according to claim 11, wherein sulforaphane is present in the composition in a concentration which is at least 20% w/w.

14. A composition according to any preceding claim, wherein sulforaphane is not part of a plant extract.

15. A composition according to any preceding claim, which comprises erucic acid in a concentration of not more than 5% w/w.

16. A composition according to any preceding claim, wherein the fatty acid or fatty acids are selected from Butyric acid, Valeric acid, Caproic acid, Enanthic acid, Caprylic acid, Pelargonic acid, Capric acid, Undecylic acid, Lauric acid, Tridecylic acid, Myristic acid, Pentadecylic acid, Palmitic acid, Margaric acid, Stearic acid, Nonadecylic acid, Arachidic acid, Heneicosylic acid, Behenic acid, Tricosylic acid, Lignoceric acid, Pentacosylic acid, Cerotic acid, Carboceric acid, Montanic acid, Nonacosylic acid, Melissic acid, Hentriacontylic acid, Lacceroic acid, Psyllic acid, Geddic acid, Ceroplastic acid, Hexatriacontylic acid, Heptatriacontylic acid, Octatriacontylic acid, Nonatriacontylic acid, Tetracontylic acid, Palmitoleic acid, cis-Vaccenic acid, Linoleic acid, Linoelaidic acid, γ-Linolenic acid, Oleic acid, Elaidic acid, cis-9-eicosenoic acid, cis-11-eicosenoic acid, Mead acid, Hexadecatrienoic acid, α-Linolenic acid (ALA), Stearidonic acid, Eicosadienoic acid, Eicosatrienoic acid, Eisocatetraenoic acid, Eicosapentaenoic acid (EPA), Heneicosapentaenoic acid, Docosapentaenoic acid (Clupanodonic acid), Docosahexaenoic acid (DHA), Tetracosapentaenoic acid, Tetracosahexaenoic acid, Dihomo-γ-linolenic acid, Arachidonic acid, Paullinic acid, Gondoic acid, Mead acid, Cervonic acid, Docosadienoic acid, Adrenic acid, Osbind acid (Docosapentaenoic acid), Tetracosatetraenoic acid, Tetracosapentaenoic acid, Docosatetraenoic acid, Nervonic acid.

17. A composition according to any preceding claim, wherein the fatty acid or fatty acids are selected from Lauric acid, Myristic acid, Palmitic acid, Stearic acid, Arachidic acid, Behenic acid, Palmitoleic acid, cis-Vaccenic acid, Linoleic acid, γ-Linolenic acid, Oleic acid, cis-9-eicosenoic acid, cis-11-eicosenoic acid, α-Linolenic acid (ALA), Stearidonic acid, Eisocatetraenoic acid, Eicosapentaenoic acid (EPA), Docosapentaenoic acid (Clupanodonic acid), Docosahexaenoic acid (DHA), Dihomo-γ-linolenic acid, Arachidonic acid, Nervonic acid.

18. A composition according to any preceding claim, wherein at least one of the fatty acid or fatty acids are selected from omega-3 fatty acids.

19. A composition according to any preceding claim, wherein at least one of the fatty acid or fatty acids are selected from omega-6 fatty acids.

20. A composition according to any preceding claim, wherein the fatty acid or fatty acids have at least 16 carbon atoms in the alkyl chain.

21. A composition according to any preceding claim, wherein the fatty acid or fatty acids have from 16 to 22 carbon atoms in the alkyl chain.

22. A composition according to any preceding claim, wherein the fatty acid or fatty acids are unsaturated fatty acids.

23. A composition according to any of the preceding claims, wherein only one type of fatty acid is present.

24. A method of providing sulforaphane in a stabilized form, comprising preparing a composition as described in claims 1-23.

25. Use of a composition according to claims 1-23 in the preparation of a nutraceutical, cosmetic or pharmaceutical composition.
